(19) United States
(12) Plant Patent Application Publication
(10) Pub. No.: US 2007/0050872 P1
(43) Pub. Date: Mar. 1, 2007

(54) Pineapple lily plant named 'Eucomis 155'

(76) Inventor: Peter Beckman, Santa Cruz, CA (US)

Correspondence Address: JONDLE & ASSOCIATES P.C., 858 HAPPY CANYON ROAD SUITE 230, CASTLE ROCK, CO, 80108, US

(21) Appl. No.: 11/214,263

(22) Filed: Aug. 29, 2005

(51) Int. Cl. A01H 5/00

(52) U.S. Cl. PLT/263

(57) ABSTRACT

A pineapple lily plant particularly distinguished by a short, compact plant habit, a short flower stalk, deep purple-lavender flowers, and floriferousness is described.

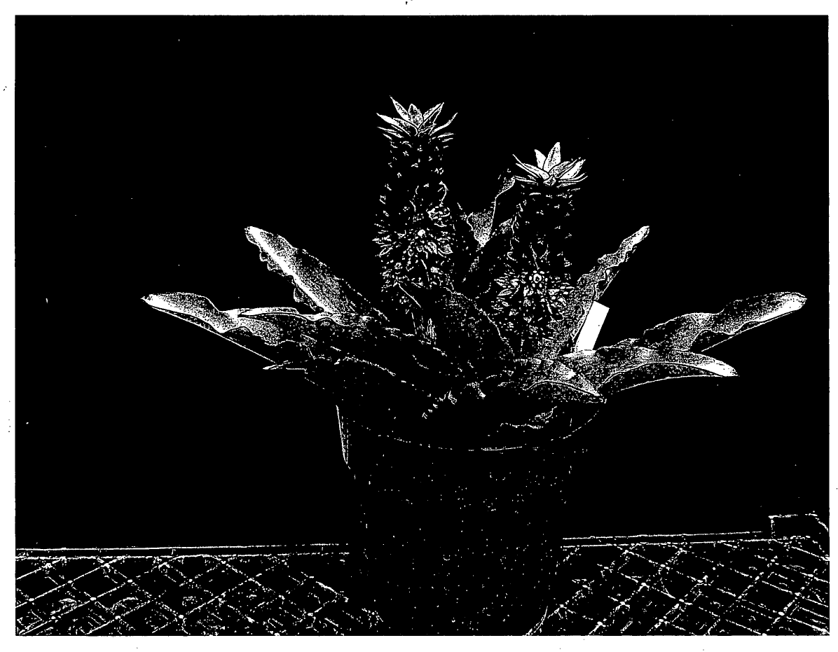

### GENUS AND SPECIES

Eucomis vandermerwei

### VARIETY DENOMINATION

‘Eucomis 155’

### BACKGROUND OF THE NEW PLANT

The present invention comprises a new and distinct cultivar of pineapple lily, botanically known as Eucomis vandermerwei, and hereinafter referred to by the cultivar name ‘Eucomis 155’. The new cultivar was an individual selection, asexually reproduced from vegetative cuttings and tissue culture resulting from the cross of a single plant of the female parent, Eucomis vandermerwei (unpatented), and the male parent, an individual plant of E. vandermerwei ‘Hawkfall Hybrids’ (patent status unknown). The first generation hybrid was obtained in 1992 and an individual selection, No. 155, was made from this first generation hybrid population. This selection was from a cross of an inbred-dwarf species, Eucomis vandermerwei, which was grown from seed, ATCC Accession No. 3454, and an unknown individual in the same species obtained from seed from Natal, South Africa and designated ‘Hawkfall Hybrids’.

‘Eucomis 155’ is a product of a planned breeding program intended to create new pineapple lily plants that are short, compact, fragrant, and brightly colored.

The new cultivar was created in Moss Landing, Calif., and has been asexually reproduced repeatedly by vegetative cuttings and tissue culture in Moss Landing, Calif. for two or more generations. The present invention has been found to retain its distinctive characteristics through successive propagations.

### SUMMARY OF THE INVENTION

The following are the most outstanding and distinguishing characteristics of this new cultivar when grown under normal horticultural practices in a brightly lit greenhouse or in an open field.
1. A short plant with a compact and full plant habit;
2. A short flower stalk;
3. Fragrant;
4. Bright and deeply saturated purple-lavender flowers; and
5. Principally plain glossy foliage (with a very faint stippled overlay at the bottom under-third of the leaf).

### DESCRIPTION OF PHOTOGRAPH

This new pineapple lily plant is illustrated by the accompanying photograph which show blooms, buds, and foliage of the plant. The colors shown are as true as can be reasonably obtained by conventional photographic procedures.

The accompanying photograph shows the overall plant habit, foliage, immature blooms, and mature blooms.

### DESCRIPTION OF THE NEW CULTIVAR

The following detailed descriptions set forth the distinctive characteristics of ‘Eucomis 155’. The data that define these characteristics were collected from asexual reproductions carried out in Moss Landing, Calif. The plant history was taken on two-year-old plants based on a 2-2.25 inch-diameter bulb under Moss Landing, Calif. summer conditions. Color references are primarily to the RHS Colour Chart of The Royal Horticultural Society of London (RHS) (2001).
Detailed botanical description:
Classification:
   Botanical.—Eucomis vandermerwei×‘Hawkfall Hybrids’.
   Common name.—Pineapple lily.
   Parentage.—Female parent: An unnamed plant of Eucomis vandermerwei (unpatented) Male parent: An unknown individual plant of Eucomis vandermerwei ‘Hawkfall Hybrids’ (patent status unknown).
Size:
   Form.—Compact with leaves arranged more or less symmetrically below the flower stocks; the overall effect of the flower stock is columnar with a topknot of leaves, somewhat like a pineapple.
   Height above the soil.—4.0-13.0 inches for a 2-2.25 inch bulb, depending on greenhouse conditions.
   Height of top of inflorescence above the soil.—6.0-13.0 inches depending on growing conditions.
   Diameter of plant.—4.0-9.0 inches.
Stems:
   Length.—5.0-13.0 inches.
   Diameter.—¼-½ inches.
   Color.—RHS 138C (medium-green) to RHS 138B (medium-green) with purple speckling up the entire stem to just below the florets.
   Texture.—Smooth.
Leaves:
   Arrangement.—More or less symmetrical below the flower stalks; leaves arise from a common base meristem as in a true bulb, where some bulbs in larger sizes have multiple base meristems.
   Quantity.—Varies, but 16-25 is typical of a 2-inch bulb grown under bright greenhouse conditions.
   Size.—Width: 1¼-2½ inches Length: 4.0-9.0 inches in direct sunlight, but shaded plants and larger bulbs have longer leaves.
   Shape.—Lanceolate.
   Color.—Upper surface: RHS 137A (green) to RHS 137B (green) with an RHS 59D (slight purple) margin Lower surface: RHS 137A (green) to RHS 137B (green) with an RHS 59D (slight purple) margin; at bottom third of the leaf base, there is faint stippling.
   Apex.—Acute.
   Base.—Contiguous with lanceolate leaf.
   Margin.—Entire and continuous with a slight undulation of the edge.
   Venation.—Parallel with slightly impressed central vein.
   Surface texture.—Smooth and slightly glossy.
Topknot:
   Size.—Individual leafy bracts are 1.0-1.5 inches in length and ¼-⅜ inches in width, making the topknot approximately 2.0-3.0 inches in both diameter and height.
   Color.—RHS 137A to RHS 137B with an RHS 59D (slight purple) margin.
   Form.—Leafy bracts of 10 to 30 bracts are arranged more or less symmetrically, radiating from the apex and resembling a pineapple topknot atop the inflorescence stalk.
   Individual leafy bract descriptors.—Essentially leaf-like, but miniature; the overall shape is lanceolate, the apex is apiculate and the base is attenuate; the margin is entire, continuous and largely smooth and no more than 10% of the leafy bracts have an RHS 59D (purple) margin from the base to the bract tip.
Bulb:
   Size.—1.5-3.5 inches in diameter for a flowering and marketable bulb.
   Color.—RHS 200A to RHS 200C (brown) outer leaf scales.
   Shape.—Non-tunicate (typical of an onion-type, true bulb form).
Inflorescence:
   Time to produce a finished flowering plant.—85-110 days.
   Number of flowers per bulb.—One or more inflorescences depending on the bulb size.
   Lastingness of individual flowers on the plant.—7-21 days.
   Fragrance.—Similar to that of coconut oil.
Bud:
   Form.—Elliptic (pomegranate seed-like).
   Size.—¼-⅝ inch in length and ¼-⅜ inch in width.
   Color.—A deep glossy purple-lavender, RHS 59A to RHS 59B.
Flower:
   Form.—Individual flowers are clustered symmetrically around the top 33%-50% of one-to-multiple inflorescences protruding at foliage level even with foliage; flowers open from the bottom to the top of the stem.
   Diameter.—Individual flowers are ½ to ¾ inch when open and clustered around the stalk.
   Inflorescence.—Typically from 9-15 inches in height and 1½-2¼ inches in width.
   Perianth lobes.—Number: 6 perianth lobes subtended by size Color: Upper surface: RHS 59A to RHS 59B on younger flowers and RHS 59D to RHS 60D for older flowers Lower surface: RHS 59A to RHS 59B on younger flowers and RHS 59D to RHS 60D for older flowers, but slightly darker Shape: Ovate Apex: Acute Base: Broad Margin: Straight, smooth and entire Texture: Smooth.
Reproductive organs:
   Androecium.—Number of anthers: 6 Filament color: RHS 58D (red-purple) at base to 59D (red-purple) at tip. Filament length: ¼-⅜ inch Pollen color: RHS 4A to RHS 5B (pale yellow to ivory) Pollen amount: Slight.
   Gynoecium.—Pistil number: 1 Pistil length: ⅛-⅜ inch Stigma color: RHS 59D to RHS 60D (red-purple) Stigma shape: Cone-shaped Style color: RHS 158D (yellow-white) to RHS 155D from base to tip Style length: 3/32-1 1/32 inch Style shape: Pyramidal cone.
Fruit/seed set: At maturity, the plant sets a dried, papery, flower-sized pod holding one to multiple seeds.
Disease and insect resistance: Pests and diseases are few; mealybugs can occasionally harbor deep in the bulb scales; Pythium can attack roots.

### Comparison with Known Cultivars

Cultivar ‘Eucomis 155’ differs from the female parent, an unnamed individual plant of Eucomis vandermerwei (unpatented) in that ‘Eucomis 155’ has more leaves that are ½ to 4½ inches longer. The deep green leaves of ‘Eucomis 155’ have purple-lavender speckling on the back only and are glossy, while the leaves of the female parent have heavy blackish-mauve and russet marks on the front and back of the leaves, and have a matte-finish. The flower color of ‘Eucomis 155’ is brighter with a more saturated lavender, and the stems are dotted with purple-lavender marks rather than the deep blackish-mauve and burgundy spots of the female parent. Additionally, ‘Eucomis 155’ is more floriferous than the female parent, having one or more flowers per bulb.

Cultivar ‘Eucomis 155’ differs from the commercial comparison variety, a typical unnamed plant of Eucomis cosmosa (unpatented) in that ‘Eucomis 155’ has shorter stems and leaves, and the flower stem is half as long as that of a typical unnamed plant of Eucomis cosmosa. Additionally, ‘Eucomis 155’ has bright, deeply saturated purple-lavender flowers while a typical unnamed plant of Eucomis cosmosa has creamy pink flowers.

Cultivar ‘Eucomis 155’ differs from the male parent (patent status unknown) in that ‘Eucomis 155’ is more floriferous and has a darker and more saturated purple flower color than the male parent.

I claim:

1. A new and distinct cultivar of Eucomis plant as shown and described herein.
